(19) United States
(12) Patent Application Publication
(10) Pub. No.: US 2008/0058613 A1
(43) Pub. Date: Mar. 6, 2008

(54) Method and System for Providing Fracture/No Fracture Classification

(75) Inventors: Philipp Lang, Lexington, MA (US); Daniel Steines, Palo Alto, CA (US); Claude Arnaud, Mill Valley, CA (US); Siau-Way Liew, Pinole, CA (US); Rene Vargas-Voracek, Sunnyvale, CA (US)

Correspondence Address: BROMBERG & SUNSTEIN LLP, 125 SUMMER STREET, BOSTON, MA, 02110-1618, US

(73) Assignee: IMAGING THERAPEUTICS, INC., Foster City, CA (US)

(21) Appl. No.: 11/855,939

(22) Filed: Sep. 14, 2007

Related U.S. Application Data
(63) Continuation-in-part of application No. 10/944,478, filed on Sep. 17, 2004.
(63) Continuation-in-part of application No. 11/228,126, filed on Sep. 16, 2005.
(60) Provisional application No. 60/825,764, filed on Sep. 15, 2006.
(60) Provisional application No. 60/503,916, filed on Sep. 19, 2003.
(60) Provisional application No. 60/610,447, filed on Sep. 16, 2004.

(51) Int. Cl. A61B 5/00

(52) U.S. Cl. 600/300

(57) ABSTRACT

A method of classifying fracture risk for a patient is presented. The method includes determining a fracture index of the patient. Either a fracture classification or a non-fracture classification is assigned to the patient based, at least in part, on the fracture index. A confidence level of the assigned classification is determined.

### CROSS-REFERENCE TO RELATED APPLICATIONS

This application claims the benefit of U.S. Application Ser. No. 60/825,764, filed Sep. 15, 2006. This application is also a continuation-in-part of U.S. application Ser. No. 10/944,478, filed Sep. 17, 2004, which in turn claims the benefit of U.S. provisional application Ser. No. 60/503,916, filed Sep. 19, 2003. This application is also a continuation-in-part of U.S. application Ser. No. 11/228,126, filed Sep. 16, 2005, which in turn claims the benefit of U.S. provisional application Ser. No. 60/610,447, filed Sep. 16, 2004. Each of the above-described documents is incorporated by reference herein in its entirety.

### TECHNICAL FIELD

The present invention relates to analysis of bone for determining risk of fracture and more particularly, to a system and method for conveying information pertaining to bone fracture/no fracture classification.

### BACKGROUND

Osteoporosis is among the most common conditions to affect the musculoskeletal system, as well as a frequent cause of locomotor pain and disability. Osteoporosis can occur in both human and animal subjects (e.g. horses). Osteoporosis (OP) occurs in a substantial portion of the human population over the age of fifty. The National Osteoporosis Foundation estimates that as many as 44 million Americans are affected by osteoporosis and low bone mass. In 1997 the estimated cost for osteoporosis related fractures was $13 billion. That figure increased to $17 billion in 2002 and is projected to increase to $210-240 billion by 2040. Currently it is expected that one in two women over the age of 50 will suffer an osteoporosis-related fracture.

In predicting skeletal disease and osteoporosis, and particularly the risk of bone fracture, a doctor and/or a patient may be presented with a large amount of information. This information should be presented to the doctor and/or the patient in a manner that is easily understood, and in a manner that eases the therapeutic decision making process.

### SUMMARY

In accordance with one embodiment of the invention, a method of classifying fracture risk for a patient is presented. The method includes determining a fracture index of the patient. Either a fracture classification or a non-fracture classification is assigned to the patient based, at least in part, on the fracture index. A confidence level of the assigned classification is determined.

In accordance with another embodiment of the invention, a computer program product for use on a computer system for classifying fracture risk for a patient is presented. The computer program product includes a computer usable medium having computer readable program code thereon. The computer readable program code includes: computer code for determining a fracture index of the patient; computer code for determining one of a fracture classification and a non-fracture classification of the patient based, at least on the fracture index; and computer code for determining a confidence level of the determined classification.

In accordance with another embodiment of the invention, a system for classifying fracture risk for a patient is presented. The system includes a controller. The controller determines a fracture index of the patient. Either a fracture classification or a non-fracture classification of the patient is assigned by the controller based, at least on the fracture index. A confidence level of the assigned fracture classification is determined by the controller.

In related embodiments of the invention, the fracture index may be based, at least in part, on at least one of, or a combination of, bone mineral density, bone micro-structure, bone macro-anatomy, and bone biomechanics. The fracture index may be based, at least in part, on trabecular bone micro-structure. Determining one of a fracture classification and a non-fracture classification may include determining a threshold fracture index value. Determining a confidence level of the determined classification may include determining a probability of making a correct classification given the fracture index of the patient. The fracture index, the determined classification, and/or the confidence level may be displayed, or a report may be generated, that includes the fracture index, the determined classification, and/or the confidence level.

These and other embodiments of the present invention will readily occur to those of ordinary skill in the art in view of the disclosure herein.

### BRIEF DESCRIPTION OF THE DRAWINGS

The foregoing features of the invention will be more readily understood by reference to the following detailed description, taken with reference to the accompanying drawings, in which:

FIG. 1 is a flowchart illustrating a method for classifying fracture risk for a patient, in accordance with an embodiment of the invention;

FIG. 2 is a flowchart illustrating a method for determining the fracture index, in accordance with an embodiment of the invention;

FIG. 3 is a plot that includes the fracture index value, determined fracture classification, as well as the confidence level of the classification, in accordance with one embodiment of the invention; and

FIG. 4 is an exemplary report that includes the fracture index value, determined fracture classification, as well as the confidence level of the classification, in accordance with one embodiment of the invention.

### DETAILED DESCRIPTION

In illustrative embodiments, a system and method of classifying fracture risk for a patient is presented. The method may include, for example, determining a fracture index of the patient. Based, at least in part, on the fracture index, a fracture classification or a non-fracture classification is assigned. A confidence level of the assigned fracture classification is determined. The fracture index, the assigned fracture classification and/or the confidence level may be displayed and/or provided in a report. Details of illustrative embodiments are discussed below.

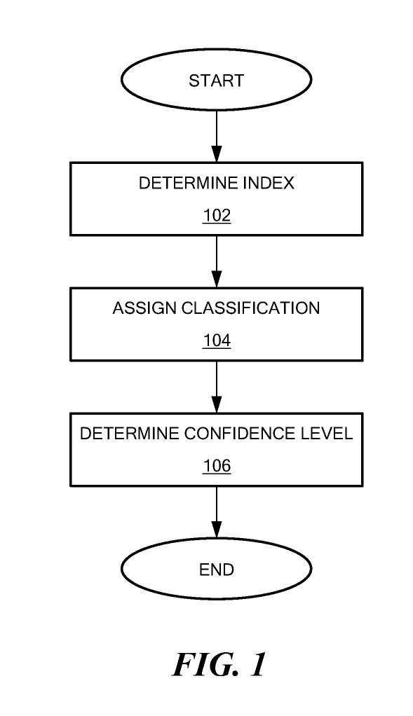

FIG. 1 is a flowchart illustrating a method for classifying fracture risk for a patient, in accordance with an embodiment of the invention. It is to be understood that the methodology shown in FIG. 1 may be used to classify risks other than fracture risk.

An index, such as a fracture index of the patient, is determined, step 102. Illustratively, the fracture index is a value pertinent to bone fracture risk that may be determined based, at least in part, on at least one of bone mineral density, bone micro-structure, bone macro-anatomy, and bone biomechanic parameters and/or measurements (for more detail, see, for example, U.S. application Ser. No. 10/944,478 (published application 20050148860), U.S. application Ser. No. 11/228,126 (published application 20060062442), and U.S. application Ser. No. 10,753,976 (published application 20040242987), each of which is incorporated herein by reference). In preferred embodiments, the fracture index may be a combination of bone mineral density, bone micro-structure, bone macro-anatomy, and bone biomechanic parameters and/or measurements. For example, the fracture index may be obtained from combining both macro and micro structural measurements from the femoral bone regions of hip radiographs using an algorithm defined through optimization and using cross-validation data.

Parameters and measurements that may be used in calculating the fracture index are shown in tables 1-3. As will be appreciated by those of skill in the art, the parameters and measurements shown in Tables 1, 2 and 3 are provided for illustration purposes and are not intended to be limiting. It will be apparent that the terms micro-structural parameters, micro-architecture, micro-anatomic structure, micro-structural and trabecular architecture may be used interchangeably. In addition, other parameters and measurements, ratios, derived values or indices can be used to extract quantitative and/or qualitative information without departing from the scope of the invention. See, e.g., co-owned International Application WO 02/30283, which is incorporated herein by reference, in its entirety. Extracted structures typically refer to simplified or amplified representations of features derived from images. An example would be binary images of trabecular patterns generated by background subtraction and thresholding. Another example would be binary images of cortical bone generated by applying an edge filter and thresholding. The binary images can be superimposed on gray level images to generate gray level patterns of structure of interest.

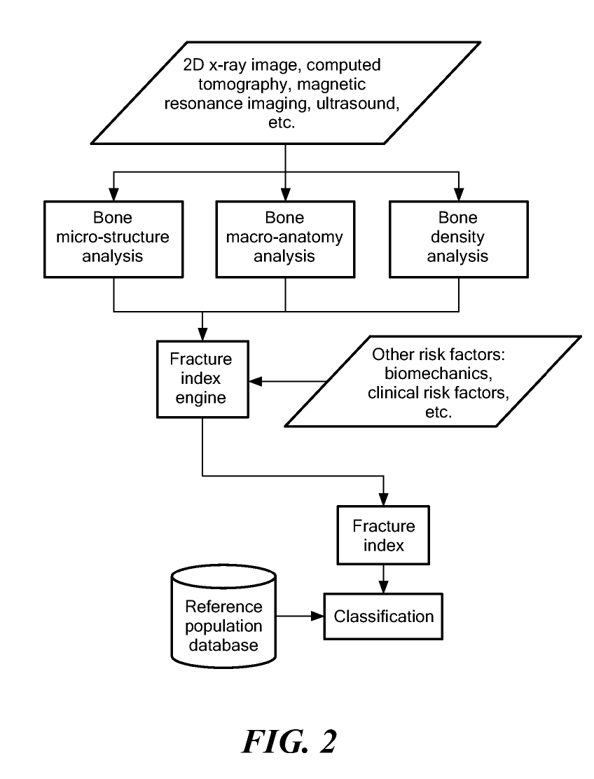

The flowchart shown in FIG. 2 depicts exemplary steps and information that can be used to determine the fracture index, in accordance with various embodiments of the invention. A 2D or 3D digital image (e.g., digitized radiographs, digital detector radiograph, computed tomography, magnetic resonance tomography etc.) including bone is taken using standard techniques.

The image is analyzed using image processing algorithms to evaluate bone micro-structure, bone density and/or bone macro-architecture.

Finally, the fracture index may be generated by combining the results from the bone micro-structure analysis, the bone density analysis and/or the bone macro-architecture analysis, optionally in combination with other risk factors. The combination may be performed, for example, using linear combinations, weighted averages or likelihood ratios.

In various embodiments of the invention, one or more measurements pertaining to, without limitation, bone mineral density, bone architecture or structure, macro-anatomy, and/or bone biomechanics, may be generated from two or more x-ray beam rotation angles. The x-rays may be generated, without limitation, by a conventional radiography unit, a conventional tomography unit (CT scan), or a digital radiography unit (e.g., digital radiography (DR) or computed radiography (CR) systems). If a DR or CR system is implemented, images may be obtained from multiple rotation angles so as to allow tomographic reconstruction.

The use of multiple x-ray beam rotation angles advantageously may be used to identify anatomical landmarks more reliably. Reproducibility may be improved. Furthermore, the use of multiple x-ray beam rotation angles may be used for semi or true three-dimensional and/or volume assessments.

Referring back to FIG. 1, the patient is next assigned, without limitation, either a fracture classification or a non-fracture classification based, at least in part, on the fracture index, step 104. The classification of a patient into fracture or non-fracture may be performed by comparing the fracture index to a threshold level value. The threshold level value may be defined by preselected sensitivity and specificity performance parameters obtained from a reference (optimization/cross-validation) data set.

A confidence level of the determined classification (e.g., either fracture classification or non-fracture classification) is then determined, step 106. For example, the confidence level of a fracture/no-fracture classification may be defined as the probability of making the correct classification given an index value and may be estimated from probabilities that can be directly estimated from result data (available information) by applying Bayes' theorem (see, for example, J. Berger. Statistical Decision Theory and Bayesian Analysis. Springer Series in Statistics. 1993; and A. Papoulis, S. U. Pillai. Probability Random Variables and Stochastic Processes. McGraw-Hill. Fourth Ed. 2001, each of which is incorporated by reference in its entirety):

$$P ( Correct Classification \vert Fracture Index ) = \frac{P ( Fracture Index \vert Correct Classification ) \cdot P ( Correct Classification )}{P ( Fracture Index )} \qquad ( 1 )$$

The first term in the numerator on the right hand side of the equation 1, represents the likelihood of a given Fracture Index value, considering (conditioned to) available information in which the classification was correct. The second term in the numerator represents the probability of making a correct classification and the term in the denominator represents the probability of a given fracture index value. The terms on the right hand side of the equation may be estimated from cross-validation data (available test and validation data) assuming that the cross-validation data is representative of the target population.

There are several possible methods for estimating/defining the terms on the right hand side of equation 1 (see, for example B. W. Silverman. Density Estimation for Statistics and Data Analysis. Chapman & Hall, 1986, which incorporated herein by reference. One method for estimating the terms on the right hand side is through histograms or plots of the number of cases for which the fracture index is within each of a set of contiguous ranges of values. Another method is by assuming a specific parametric form, e.g. a Normal/Gaussian distribution, for the fracture index, and estimate the corresponding parameters from the cross-validation data.

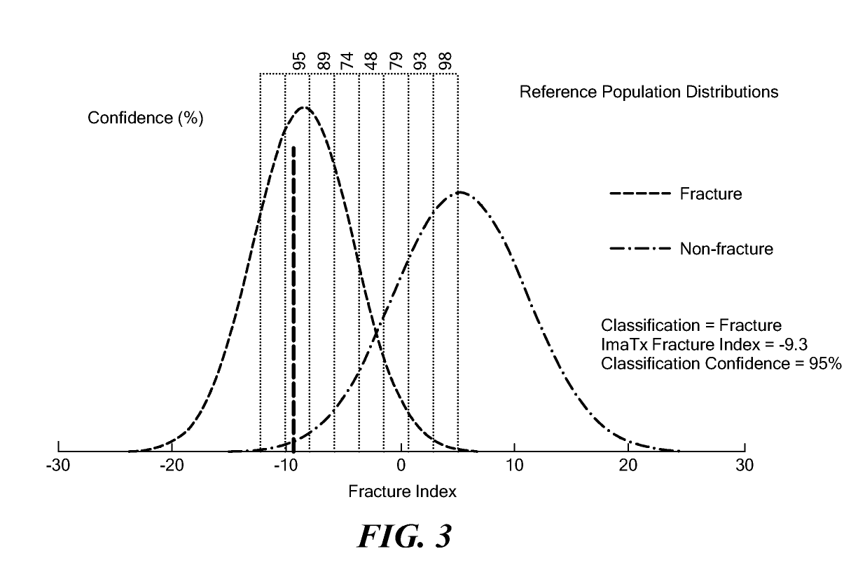

The fracture index value, determined fracture classification, as well as the confidence level of the classification can then be shown on a display and/or included in a generated report, as shown in the plot of FIG. 3, in accordance with an embodiment of the invention. Reference population information (that may be represent, for example, by a bell curve) may also be provided. Thus, the doctor or patient can make a more informed decision regarding future therapeutic treatment.

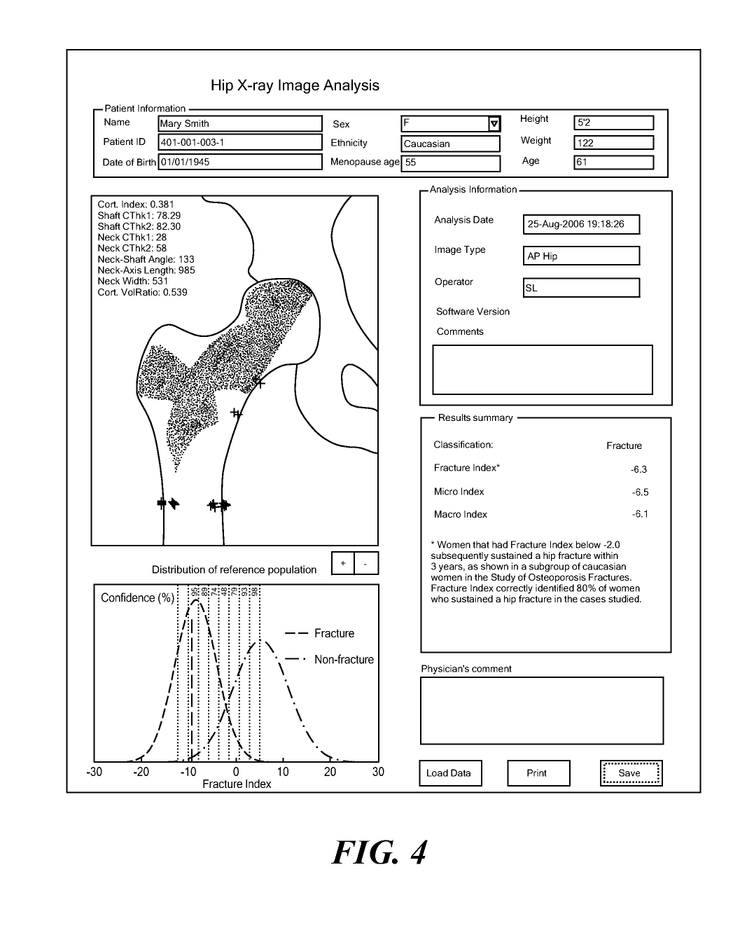

FIG. 4 is an exemplary report that includes the fracture index value, determined fracture classification, as well as the confidence level of the classification, in accordance with one embodiment of the invention. As can be seen, illustrations showing structure, a results summary, analysis and patient information may be added to the report.

TABLE 1
Representative Parameters Measured with Quantitative
and Qualitative Image Analysis Methods

| PARAMETER | MEASUREMENTS |
|---|---|
| Bone density and | Calibration phantom equivalent thickness |
| microstructural | (Average intensity value of the region of interest expressed as |
| parameters | thickness of calibration phantom that would produce the equivalent |
| | intensity) |
| | Trabecular contrast |
| | Standard deviation of background subtracted ROI |
| | Coefficient of Variation of ROI (Standard deviation/mean) |
| | (Trabecular equivalent thickness/Marrow equivalent thickness) |
| | Fractal dimension |
| | Hough transform |
| | Fourier spectral analysis |
| | (Mean transform coefficient absolute value and mean spatial first |
| | moment) |
| | Predominant orientation of spatial energy spectrum |
| | Trabecular area |
| | (Pixel count of extracted trabeculae) |
| | Trabecular area/Total area |
| | Trabecular perimeter |
| | (Count of trabecular pixels with marrow pixels in their neighborhood, |
| | proximity or vicinity) |
| | Trabecular distance transform |
| | (For each trabecular pixel, calculation of distance to closest marrow |
| | pixel) |
| | Marrow distance transform |
| | (For each marrow pixel, calculation of distance to closest trabecular |
| | pixel) |
| | Trabecular distance transform regional maximal values (mean, min., |
| | max, std. Dev). |
| | (Describes thickness and thickness variation of trabeculae) |
| | Marrow distance transform regional maximal values (mean, min., max, |
| | std. Dev) |
| | Star volume |
| | (Mean volume of all the parts of an object which can be seen |
| | unobscured from a random point inside the object in all possible |
| | directions) |
| | Trabecular Bone Pattern Factor |
| | (TBPf = (P1 − P2)/(A1 − A2) where P1 and A1 are the perimeter |
| | length and trabecular bone area before dilation and P2 and A2 |
| | corresponding values after a single pixel dilation, measure of |
| | connectivity) |
| | Connected skeleton count or Trees (T) |
| | Node count (N) |
| | Segment count (S) |
| | Node-to-node segment count (NN) |
| | Node-to-free-end segment count (NF) |
| | Node-to-node segment length (NNL) |
| | Node-to-free-end segment length (NFL) |
| | Free-end-to-free-end segment length (FFL) |
| | Node-to-node total struts length (NN.TSL) |
| | Free-end-to-free-ends total struts length(FF.TSL) |
| | Total struts length (TSL) |
| | FF.TSL/TSL |
| | NN.TSL/TSL |
| | Loop count (Lo) |
| | Loop area |
| | Mean distance transform values for each connected skeleton |
| | Mean distance transform values for each segment (Tb.Th) |
| | Mean distance transform values for each node-to-node segment |
| | (Tb.Th.NN) |
| | Mean distance transform values for each node-to-free-end segment |
| | (Tb.Th.NF) |
| | Orientation (angle) of each segment |
| | Angle between segments |
| | Length-thickness ratios (NNL/Tb.Th.NN) and (NFL/Tb.Th.NF) |
| | Interconnectivity index (ICI) ICI = (N * NN)/(T * (NF + 1)) |
| Cartilage and | Total cartilage volume |
| cartilage | Partial/Focal cartilage volume |
| defect/diseased | Cartilage thickness distribution (thickness map) |
| cartilage parameters | Mean cartilage thickness for total region or focal region |
| | Median cartilage thickness for total region or focal region |
| | Maximum cartilage thickness for total region or focal region |
| | Minimum cartilage thickness for total region or focal region |
| | 3D cartilage surface information for total region or focal region |
| | Cartilage curvature analysis for total region or focal region |
| | Volume of cartilage defect/diseased cartilage |
| | Depth of cartilage defect/diseased cartilage |
| | Area of cartilage defect/diseased cartilage |
| | 2D or 3D location of cartilage defect/diseased cartilage in articular |
| | surface |
| | 2D or 3D location of cartilage defect/diseased cartilage in |
| | relationship to weight-bearing area |
| | Ratio: diameter of cartilage defect or diseased cartilage/thickness of |
| | surrounding normal cartilage |
| | Ratio: depth of cartilage defect or diseased cartilage/thickness of |
| | surrounding normal cartilage |
| | Ratio: volume of cartilage defect or diseased cartilage/thickness of |
| | surrounding normal cartilage |
| | Ratio: surface area of cartilage defect or diseased cartilage/total |
| | joint or articular surface area |
| | Ratio: volume of cartilage defect or diseased cartilage/total cartilage |
| | volume |
| Other articular | Presence or absence of bone marrow edema |
| parameters | Volume of bone marrow edema |
| | Volume of bone marrow edema normalized by width, area, size, |
| | volume of femoral condyle(s)/tibial plateau/patella - other bones |
| | in other joints |
| | Presence or absence of osteophytes |
| | Presence or absence of subchondral cysts |
| | Presence or absence of subchondral sclerosis |
| | Volume of osteophytes |
| | Volume of subchondral cysts |
| | Volume of subchondral sclerosis |
| | Area of bone marrow edema |
| | Area of osteophytes |
| | Area of subchondral cysts |
| | Area of subchondral sclerosis |
| | Depth of bone marrow edema |
| | Depth of osteophytes |
| | Depth of subchondral cysts |
| | Depth of subchondral sclerosis |
| | Volume, area, depth of osteophytes, subchondral cysts, subchondral |
| | sclerosis normalized by width, area, size, volume of femoral |
| | condyle(s)/tibial plateau/patella - other bones in other joints |
| | Presence or absence of meniscal tear |
| | Presence or absence of cruciate ligament tear |
| | Presence or absence of collateral ligament tear |
| | Volume of menisci |
| | Ratio of volume of normal to torn/damaged or degenerated meniscal |
| | tissue |
| | Ratio of surface area of normal to torn/damaged or degenerated |
| | meniscal tissue |
| | Ratio of surface area of normal to torn/damaged or degenerated |
| | meniscal tissue to total joint or cartilage surface area |
| | Ratio of surface area of torn/damaged or degenerated meniscal |
| | tissue to total joint or cartilage surface area |
| | Size ratio of opposing articular surfaces |
| | Meniscal subluxation/dislocation in mm |
| | Index combining different articular parameters which can also |
| | include |
| | Presence or absence of cruciate or collateral ligament tear |
| | Body mass index, weight, height |
| | 3D surface contour information of subchondral bone |
| | Actual or predicted knee flexion angle during gait cycle |
| | (latter based on gait patterns from subjects with matching |
| | demographic data retrieved from motion profile database) |
| | Predicted knee rotation during gait cycle |
| | Predicted knee displacement during gait cycle |
| | Predicted load bearing line on cartilage surface during gait cycle and |
| | measurement of distance between load bearing line and cartilage |
| | defect/diseased cartilage |
| | Predicted load bearing area on cartilage surface during gait cycle |
| | and measurement of distance between load bearing area and |
| | cartilage defect/diseased cartilage |
| | Predicted load bearing line on cartilage surface during standing or |
| | different degrees of knee flexion and extension and measurement |
| | of distance between load bearing line and cartilage |
| | defect/diseased cartilage |
| | Predicted load bearing area on cartilage surface during standing or |
| | different degrees of knee flexion and extension and measurement |
| | of distance between load bearing area and cartilage |
| | defect/diseased cartilage |
| | Ratio of load bearing area to area of cartilage defect/diseased |
| | cartilage |
| | Percentage of load bearing area affected by cartilage disease |
| | Location of cartilage defect within load bearing area |
| | Load applied to cartilage defect, area of diseased cartilage |
| | Load applied to cartilage adjacent to cartilage defect, area of |
| | diseased cartilage |

TABLE 2
Site specific measurement of bone parameters

| Parameters specific to | All microarchitecture parameters on structures parallel to stress | |
|---|---|---|
| hip images | lines | |
| | All microarchitecture parameters on structures perpendicular to | |
| | stress lines | |
| | Geometry | |
| | Shaft angle | |
| | Neck angle | |
| | Average and minimum diameter of femur neck | |
| | Hip axis length | |
| | CCD (caput-collum-diaphysis) angle | |
| | Width of trochanteric region | |
| | Largest cross-section of femur head | |
| | Standard deviation of cortical bone thickness within ROI | |
| | Minimum, maximum, mean and median thickness of cortical | |
| | bone within ROI | |
| | Hip joint space width | |
| Parameters specific to | All microarchitecture parameters on vertical structures | |
| spine images | All microarchitecture parameters on horizontal structures | |
| | Geometry | |
| | 1. | Superior endplate cortical thickness (anterior, center, |
| | | posterior) |
| | 2. | Inferior endplate cortical thickness (anterior, center, |
| | | posterior) |
| | 3. | Anterior vertebral wall cortical thickness (superior, |
| | | center, inferior) |
| | 4. | Posterior vertebral wall cortical thickness (superior, |
| | | center, inferior) |
| | 5. | Superior aspect of pedicle cortical thickness |
| | 6. | inferior aspect of pedicle cortical thickness |
| | 7. | Vertebral height (anterior, center, posterior) |
| | 8. | Vertebral diameter (superior, center, inferior), |
| | 9. | Pedicle thickness (supero-inferior direction). |
| | 10. | Maximum vertebral height |
| | 11. | Minimum vertebral height |
| | 12. | Average vertebral height |
| | 13. | Anterior vertebral height |
| | 14. | Medial vertebral height |
| | 15. | Posterior vertebral height |
| | 16. | Maximum inter-vertebral height |
| | 17. | Minimum inter-vertebral height |
| | 18. | Average inter-vertebral height |

| | |
|---|---|
| Parameters specific to | Average medial joint space width |
| knee images | Minimum medial joint space width |
| | Maximum medial joint space width |
| | Average lateral joint space width |
| | Minimum lateral joint space width |
| | Maximum lateral joint space width |

TABLE 3
Measurements applicable on Microarchitecture and Macro-anatomical Structures

| | |
|---|---|
| Average density | Calibrated density of ROI |
| measurement | |
| Measurements on micro- | The following parameters are derived from the extracted structures: |
| anatomical structures of | Calibrated density of extracted structures |
| dental, spine, hip, knee or | Calibrated density of background |
| bone cores images | Average intensity of extracted structures |
| | Average intensity of background (area other than extracted |
| | structures) |
| | Structural contrast (average intensity of extracted structures/ |
| | average intensity of background) |
| | Calibrated structural contrast (calibrated density extracted |
| | structures/calibrated density of background) |
| | Total area of extracted structures |
| | Total area of ROI |
| | Area of extracted structures normalized by total area of ROI |
| | Boundary lengths (perimeter) of extracted normalized by total |
| | area of ROI |
| | Number of structures normalized by area of ROI |
| | Trabecular bone pattern factor; measures concavity and |
| | convexity of structures |
| | Star volume of extracted structures |
| | Star volume of background |
| | Number of loops normalized by area of ROI |
| Measurements on | The following statistics are measured from the distance transform |
| Distance transform of | regional maximum values: |
| extracted structures | Average regional maximum thickness |
| | Standard deviation of regional maximum thickness |
| | Largest value of regional maximum thickness |
| | Median of regional maximum thickness |
| Measurements on | Average length of networks (units of connected segments) |
| skeleton of extracted | Maximum length of networks |
| structures | Average thickness of structure units (average distance |
| | transform values along skeleton) |
| | Maximum thickness of structure units (maximum distance |
| | transform values along skeleton) |
| | Number of nodes normalized by ROI area |
| | Number of segments normalized by ROI area |
| | Number of free-end segments normalized by ROI area |
| | Number of inner (node-to-node) segments normalized ROI area |
| | Average segment lengths |
| | Average free-end segment lengths |
| | Average inner segment lengths |
| | Average orientation angle of segments |
| | Average orientation angle of inner segments |
| | Segment tortuosity; a measure of straightness |
| | Segment solidity; another measure of straightness |
| | Average thickness of segments (average distance transform |
| | values along skeleton segments) |
| | Average thickness of free-end segments |
| | Average thickness of inner segments |
| | Ratio of inner segment lengths to inner segment thickness |
| | Ratio of free-end segment lengths to free-end segment thickness |
| | Interconnectivity index; a function of number of inner segments, |
| | free-end segments and number of networks. |
| Directional skeleton | All measurement of skeleton segments can be constrained by |
| segment | one or more desired orientation by measuring only skeleton |
| measurements | segments within ranges of angle. |
| Watershed | Watershed segmentation is applied to gray level images. |
| segmentation | Statistics of watershed segments are: |
| | Total area of segments |
| | Number of segments normalized by total area of segments |
| | Average area of segments |
| | Standard deviation of segment area |
| | Smallest segment area |
| | Largest segment area |

The present invention may be embodied in many different forms, including, but in no way limited to, computer program logic for use with a processor (e.g., a microprocessor, microcontroller, digital signal processor, or general purpose computer), programmable logic for use with a programmable logic device (e.g., a Field Programmable Gate Array (FPGA) or other PLD), discrete components, integrated circuitry (e.g., an Application Specific Integrated Circuit (ASIC)), or any other means including any combination thereof.

Computer program logic implementing all or part of the functionality previously described herein may be embodied in various forms, including, but in no way limited to, a source code form, a computer executable form, and various intermediate forms (e.g., forms generated by an assembler, compiler, linker, or locator.) Source code may include a series of computer program instructions implemented in any of various programming languages (e.g., an object code, an assembly language, or a high-level language such as Fortran, C, C++, JAVA, or HTML) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The computer program may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device ( e.g., a diskette or fixed disk), an optical memory device (e.g., a CD-ROM), a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies, networking technologies, and internetworking technologies. The computer program may be distributed in any form as a removable storage medium with accompanying printed or electronic documentation (e.g., shrink wrapped software or a magnetic tape), preloaded with a computer system (e.g., on system ROM or fixed disk), or distributed from a server or electronic bulletin board over the communication system (e.g., the Internet or World Wide Web.)

Hardware logic (including programmable logic for use with a programmable logic device) implementing all or part of the functionality previously described herein may be designed using traditional manual methods, or may be designed, captured, simulated, or documented electronically using various tools, such as Computer Aided Design (CAD), a hardware description language (e.g., VHDL or AHDL), or a PLD programming language (e.g., PALASM, ABEL, or CUPL.)

Although various exemplary embodiments of the invention have been disclosed, it should be apparent to those skilled in the art that various changes and modifications can be made which will achieve some of the advantages of the invention without departing from the true scope of the invention. These and other obvious modifications are intended to be covered by the appended claims.

What is claimed is:

1. A method of classifying fracture risk for a patient, the method comprising:
determining a fracture index of the patient;
determining one of a fracture classification and a non-fracture classification of the patient based, at least in part, on the fracture index; and
determining a confidence level of the determined classification.

2. The method of claim 1, wherein the fracture index is based, at least in part, on at least one of bone mineral density, bone micro-structure, bone macro-anatomy, and bone biomechanics.

3. The method of claim 2, wherein the fracture index is based, at least in part, on two or more of bone mineral density, bone micro-structure, bone macro-anatomy and bone biomechanics.

4. The method of claim 1, wherein the fracture index is based, at least in part, on trabecular bone micro-structure.

5. The method of claim 1, wherein determining one of a fracture classification and a non-fracture classification includes determining a threshold fracture index value.

6. The method of claim 1, wherein determining a confidence level of the determined classification includes determining a probability of making a correct classification given the fracture index of the patient.

7. The method of claim 1, further comprising displaying the fracture index, the determined classification, and/or the confidence level.

8. The method of claim 1, further comprising generating a report that includes the fracture index, the determined classification, and/or the confidence level.

9. A computer program product for use on a computer system for classifying fracture risk for a patient, the computer program product comprising a computer usable medium having computer readable program code thereon, the computer readable program code including:
computer code for determining a fracture index of the patient;
computer code for determining one of a fracture classification and a non-fracture classification of the patient based, at least on the fracture index; and
computer code for determining a confidence level of the determined classification.

10. The computer program product according to claim 9, wherein the computer code for determining the fracture index includes determining the fracture index based, at least in part, on at least one of bone mineral density, bone micro-structure, bone macro-anatomy, and bone biomechanics.

11. The computer program product according to claim 10, wherein the computer code for determining the fracture index includes determining the fracture index based, at least in part, on two or more of bone mineral density, bone micro-structure, bone macro-anatomy and bone biomechanics.

12. The computer program product according to claim 9, wherein the computer code for determining the fracture index includes determining the fracture index based, at least in part, on trabecular bone micro-structure.

13. The computer program product according to claim 9, wherein the computer code for determining one of the fracture classification and the non-fracture classification includes determining a threshold fracture index value.

14. The computer program product according to claim 9, wherein the computer code for determining the confidence level of the determined fracture classification includes determining a probability of making a correct classification given the fracture index of the patient.

15. The computer program product according to claim 9, further comprising computer code for displaying the fracture index, the determined fracture classification, and/or the confidence level.

16. The computer program product according to claim 9, further comprising computer code for generating a report that includes the fracture index, the determined fracture classification, and/or the confidence level.

17. A system for classifying fracture risk for a patient, the system comprising:
a controller, the controller for
determining a fracture index of the patient;
determining one of a fracture classification and a non-fracture classification of the patient based, at least on the fracture index; and
determining a confidence level of the determined fracture classification.

18. The system of claim 17, wherein the fracture index is based, at least in part, on at least one of bone mineral density, bone micro-structure, bone macro-anatomy, and bone biomechanics.

19. The system of claim 18, wherein the fracture index is based, at least in part, on two or more of bone mineral density, bone micro-structure, bone macro-anatomy and bone biomechanics.

20. The system of claim 17, wherein the fracture index is based, at least in part, on trabecular bone micro-structure.

21. The system of claim 17, wherein determining one of a fracture classification and a non-fracture classification includes determining a threshold fracture index value.

22. The system of claim 17, wherein determining a confidence level of the determined fracture classification includes determining a probability of making a correct classification given the fracture index of the patient.

23. The system of claim 17, further comprising a display, wherein the controller controls the display to display the fracture index, the determined fracture classification, and/or the confidence level.

24. The system of claim 17, wherein the controller generates a report that includes the fracture index, the determined fracture classification, and/or the confidence level.
